(19) United States
(12) Patent Application Publication
(10) Pub. No.: US 2014/0058224 A1
(43) Pub. Date: Feb. 27, 2014

(54) SYSTEMS AND METHODS FOR DETECTION OF CAROTENOID-RELATED COMPOUNDS IN BIOLOGICAL TISSUE

(71) Applicants: Werner Gellermann, Salt Lake City, UT (US); Mohsen Sharifzadeh, Salt Lake City, UT (US)

(72) Inventors: Werner Gellermann, Salt Lake City, UT (US); Mohsen Sharifzadeh, Salt Lake City, UT (US)

(73) Assignee: Opticks, Inc., Salt Lake City, UT (US)

(21) Appl. No.: 13/590,961

(22) Filed: Aug. 21, 2012

(51) Int. Cl. A61B 5/1455

(52) U.S. Cl. 600/314

(57) ABSTRACT

A method for measuring and quantifying biological compounds is described. A first side of a sample is illuminated with a light source. Light transmitted from a second side of the sample is detected. The second side of the sample is opposite the first side of the sample. A result is obtained based on the detected light.

### TECHNICAL FIELD

The present invention relates generally to biomedical optics and biomedical optics-related technology. More specifically, the present invention relates to systems and methods for detection and measurement of levels of carotenoid-related compounds in biological tissue.

### BACKGROUND

Biological compounds existing in living human tissue may be used to determine information relating to a subject. For example, the presence of environmental toxins may be determined by identification of biological compounds. Biological compounds may also be used to detect the presence of a disease. For instance, the presence of antibodies may indicate that a disease has been detected by a subject's immune system. The biological compounds of interest in this patent application are carotenoid-related compounds.

Carotenoids are important plant pigments routinely ingested on a daily basis via fruit and vegetable consumption. The most prevalent carotenoids consumed in North American Diets include alpha-carotene, beta-carotene, lycopene, lutein, zeaxanthin and beta-cryptoxanthin. [“Dietary reference intakes for vitamin C, vitamin E, selenium, and carotenoids: a report of the panel on Dietary Antioxidants and Related Compounds,” Food and Nutrition Board, Institute of Medicine, National Academy of Sciences, National Academy Press, Washington, D.C. (2000)]. Carotenoids can be measured in blood, in skin, in the macular region of the human retina, and in other tissues. Blood and skin carotenoid levels are correlated with dietary intake of fruits and vegetables [Y. M. Peng, Y. S. Peng, Y. Lin, T. Moon, D. J. Roe, and C. Ritenbaugh, “Concentrations and plasma tissue diet relationships of carotenoids, retinoids, and tocopherols in humans,” Nutrition and Cancer. 23, 234-246 (1995)]. Therefore, measurements of blood and skin carotenoid levels can serve as an objective biomarker of fruit and vegetable intake. Fruit and vegetable consumption is generally regarded as an important factor for increased energy and overall good health. For example, high dietary consumption of fruits and vegetables has been associated with protection against a number of diseases, including various cancers [“Food, nutrition, physical activity, and the prevention of cancer: a global perspective,” World Cancer Research Fund, American Institution for Cancer Research, Washington, D.C. (2007)], cardiovascular disease [S. Liu, J. E. Manson, I. M. Lee, S. R. Cole, C. H. Hennekens, W. C. Willett, and J. E. Buring, “Fruit and vegetable intake and risk of cardiovascular disease: the Women's Health Study,” Am. J. Clin. Nutr. 72, 922-928 (2000)], age-related macular degeneration, and pre-mature skin aging [see, e.g., P. S. Bernstein and W. Gellermann, “Noninvasive Assessment of Carotenoids in the Human Eye and Skin,” chapter 3 in: “Carotenoids in Health and Disease,” N. I. Krinsky, S. T. Mayne, and H. Sies, (eds.), Marcel Dekker, New York, N.Y. (2004)]. Furthermore, carotenoids themselves have been speculated to be one of the anti-carcinogenic phyto-chemicals of plant foods and are thought to protect the tissue cells via optical filtering and/or antioxidant action. For all these reasons, it is compelling to develop convenient detection methodologies for carotenoids and related compounds directly in living human tissue.

The standard method for the measurement of carotenoids is based on biochemical high-performance liquid chromatography (HPLC) techniques. However, these HPLC techniques are highly invasive. They require that relatively large amounts of tissue be removed from the subject for subsequent tissue processing and analysis, which besides being painful, costly and inconvenient, also takes at least several hours to complete. In the course of these types of analyses, the tissue is damaged, if not completely destroyed. Alternatively, carotenoid concentrations can be indirectly estimated via HPLC analysis of plasma or serum. Key disadvantages again are discomfort, cost and necessity of venipuncture, which may cause participation bias since subjects may be reluctant to give blood. Furthermore, carotenoid concentrations in blood fluctuate in response to recent dietary intake, with an estimated half-life of less than 12 days for beta-carotene [C. L. Rock, M. E. Swendseid, R. A. Jacob, and R. W. McKee, “Plasma carotenoid levels in human subjects fed a low carotenoid diet,” J. Nutr. 122, 96-100 (1992)]. The situation is even worse in the human retina, where only two of the approximately half dozen carotenoid species circulating in blood, i.e. lutein and zeaxanthin, are taken up and are concentrated in this tissue. Consequently, there is at best only a very poor correlation with plasma levels for this particular tissue. In general it is necessary to develop novel, non-invasive, methods for the detection of carotenoid levels directly in the tissue of interest.

### BRIEF DESCRIPTION OF THE DRAWINGS

In order to illustrate the above and other features of the present invention, a more particular description of the invention will be rendered by reference to specific examples thereof, which are illustrated in the appended drawings. It is appreciated that these drawings depict only typical examples of the invention and are therefore not to be considered limiting of its scope. The invention will be described and explained with additional specificity and detail through the use of the accompanying drawings in which:

FIG. 1 is a graphical diagram of the absorption spectra, molecular structure and energy level scheme of major carotenoid species found in human tissue, including beta-carotene (β-carotene), zeaxanthin, lycopene, lutein and phytofluene;

FIG. 2 illustrates model absorption spectra of the main absorbing chromophores in melanin-free human skin tissue.

FIG. 3 is a block diagram illustrating one example of an absorption spectroscopy based apparatus for measuring and quantifying biological compounds in tissue;

FIG. 4 is a block diagram illustrating a more detailed example of an absorption spectroscopy based apparatus for measuring and quantifying biological compounds in tissue;

FIG. 5A illustrates light propagation through the various layers of tissue in a human finger;

FIG. 5B illustrates light propagation through the various layers of tissue in a boneless body part, such as an ear lobe or a tissue fold;

FIG. 6 illustrates typical emission spectra of two different light sources suitable for absorption measurements;

FIG. 7 illustrates the absorption spectrum of a 1 mm thick quartz cuvette filled with a methanolic carotenoid solution;

FIG. 8 illustrates transmission-derived carotenoid absorption spectra of an excised, bloodless heel skin tissue sample in the 350-540 nm wavelength region;

FIG. 9A illustrates the transmission spectrum measured for a living human finger;

FIG. 9B illustrates the absorption spectrum derived from the transmission spectrum in FIG. 9 A;

FIG. 10 illustrates the transmission-derived absorption spectra of different fingers (thumb, index and small finger) of a subject;

FIG. 11 illustrates absorption spectra of index fingers from two different subjects;

FIG. 12 illustrates the absorption spectrum of the thenar skin fold between thumb and index finger of a human subject;

FIG. 13 illustrates absorption spectra of ear lobes of two different subjects;

FIG. 14A and FIG. 14B illustrate absorption spectra from an excised rib tissue sample of a grass-fed cow;

FIG. 15 illustrates time-resolved absorption behavior of a human finger in the carotenoid absorption region, showing a modulation of the carotenoid absorption due to the heart rhythm;

FIG. 16 illustrates a flow diagram of a method for measuring and quantifying biological compounds in tissue;

FIG. 17 illustrates a more detailed flow diagram of a method for measuring and quantifying biological compounds in tissue; and

FIG. 18 is a block diagram illustrating various hardware components that may be used in a computing device.

### DETAILED DESCRIPTION

A method for measuring and quantifying biological compounds is described. A first side of a sample is illuminated with a light source. Light transmitted from a second side of the sample is detected. The second side of the sample is opposite the first side of the sample. A result is obtained based on the detected light.

Detecting the transmitted light may include using an optical detector. The sample may be skin, fibrous tissue, fat, bone, blood, cartilage or a combination thereof. The sample may be a finger, a hand, a tissue fold of an arm, a tissue fold of a breast, a tissue fold of a hand, a thenar tissue fold or an earlobe.

The light source may have an intensity that does not substantially alter biological compound levels in the sample. The light source may be a light emitting diode, a light emitting diode array, a tungsten halogen lamp or any other suitable broad band light source.

The result may be based on levels of carotenoids in the sample. The light source may generate light at a wavelength that overlaps the absorption band of carotenoids and extends into adjacent spectral regions. The result may be based on transmitted light detected in a spectral region centered at approximately 480 nm and referenced against the transmitted light in adjacent spectral regions. Obtaining a result may include analyzing the detected light to obtain a result. The result may be displayed. The result may be used to obtain an antioxidant status of the sample. Concentration levels of carotenoids in the result may be compared to concentration levels of carotenoids in normal biological tissue to assess the risk or presence of a malignancy or other disease.

The result may be based on time-resolved absorption of the sample. Obtaining the result may include analyzing the sample to determine carotenoid levels circulating in blood and carotenoid levels in the sample. Obtaining the result may include analyzing the sample to determine the level of other chromophores circulating in blood relative to their levels in the sample. The sample may be approximately a millimeter to three centimeters thick, measuring from the first side of the sample to the second side of the sample.

An apparatus for measuring and quantifying biological compounds is also described. The apparatus includes a light source that illuminates a first side of a sample. The apparatus also includes an optical detector that detects light transmitted from a second side of the sample. The second side of the sample is opposite the first side of the sample.

The apparatus may include an enclosure. The enclosure may prevent the optical detector from detecting any light not transmitted from the second side of the sample. The apparatus may include a spectrograph that analyses and quantifies the transmitted light detected at the optical detector to obtain a result.

Biological compounds in human tissue may be used to determine information relating to a subject. For example, the presence of environmental toxins may be determined using biological compounds. Biological compounds may also be used to detect the presence of a disease. For instance, the presence of antibodies may indicate that a disease has been detected by a subject's immune system.

Some biological compounds may be found in the skin and/or other tissues of the body. Detection and measurement of biological compounds may require expensive equipment, long measurement periods and/or other challenges. For example, detection of biological substances in the skin may require removing a tissue sample and performing biochemical analysis of the sample. Removing samples may cause a subject pain, while analysis may require that the sample be sent to a lab.

One example of such biological compounds are carotenoids and related compounds. Carotenoids are important ingredients for the anti-oxidant defense system of the human body. Numerous epidemiological and experimental studies have shown that a higher dietary intake of carotenoids may protect against cancer, age-related macular degeneration, pre-mature skin aging, and other pathologies associated with oxidative cell damage. Carotenoids are found in most fruits and vegetables and are not naturally produced by the human body. Thus, the finding of carotenoids in the human body indicates consumption of fruits and vegetables. As the level of consumption of fruits and vegetables increases, so does the level of carotenoids in the body.

A noninvasive method for the measurement of carotenoids and related chemical substances in biological tissue by resonance Raman spectroscopy is disclosed in U.S. Pat. No. 6,205,354 B1, the disclosure of which is incorporated by reference herein. This technique provides for a rapid, accurate and safe determination of carotenoid levels that in turn can provide diagnostic information regarding fruit and vegetable consumption, nutritional supplement uptake or it can be a marker for conditions where carotenoids or other antioxidant compounds may provide disease-related diagnostic information. In this technique, a laser or other spectrally narrow light is directed upon the tissue area of interest, such as the palm of the hand. A small fraction of the scattered light is scattered inelastically, producing the carotenoid Raman signal that is at a different frequency or corresponding wavelength than the incident laser light, and the Raman signal is collected, filtered and measured. The Raman signal can be analyzed such that the background fluorescence signal is subtracted and the result displayed and compared with known calibration standards.

A further non-invasive optical method for the non-invasive assessment of skin carotenoid levels is based on reflection spectroscopy. Particularly promising is a pressure-mediated version of reflection spectroscopy that allows one to assess skin carotenoid levels after temporal removal of interfering blood chromophores. This method is disclosed in U.S. Publication No. 2009/0306521 A1 (“Noninvasive Measurement of Carotenoids in Biological Tissue”), the disclosure of which is incorporated by reference herein. The pressure mediated reflection method holds promise as a particularly simple and inexpensive method since it does not require any narrow-band light sources for excitation. Also, it does not require relatively high-resolution spectrometers as needed for detection of the spectrally narrow Raman line features.

Optical Properties of Carotenoids and Optical Methods for their Non-Invasive Detection in Biological Tissue

Carotenoids are n-electron conjugated carbon-chain molecules and are similar to polyenes with regard to their structure and optical properties. Distinguishing features are the number, n, of the conjugated carbon double bonds (C═C bonds), the number of attached methyl side groups, and the presence and structure of attached end groups. The optical detection of carotenoids in a subject may be of particular interest to the nutritional supplement industry where the formation of the carotenoid's “wear and tear” biomarker may be monitored over time and/or may be potentially increased via supplementation. The systems and methods disclosed may also be of interest to Medical Sciences such as Ophthalmology, Neonatology, Nutrition Science and Epidemiology, where they may provide a research tool useful in investigating the correlation between carotenoid antioxidants and diseases in large subject populations.

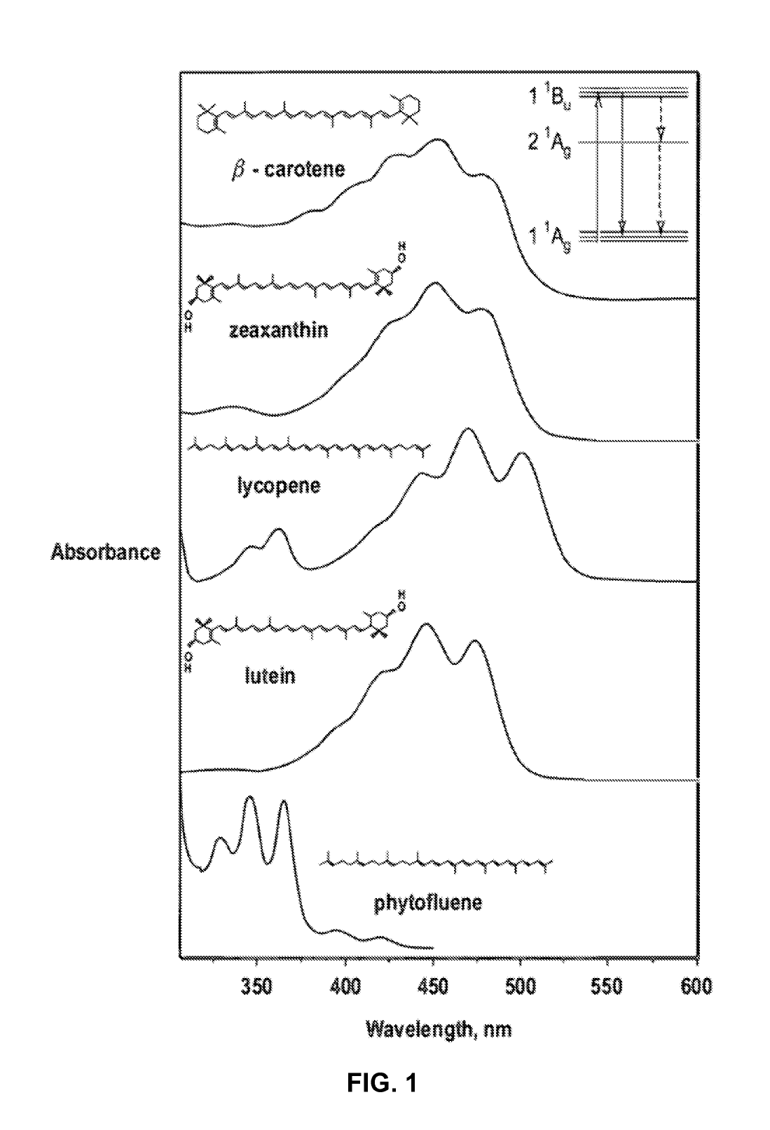

FIG. 1 is a graphical diagram of the absorption spectra, molecular structure, and energy level scheme of major carotenoid species found in human tissue, including beta-carotene (β-carotene), zeaxanthin, lycopene, lutein and phytofluene. All carotenoids feature an unusual even-parity excited state. Consequently, their absorption transitions are electric-dipole allowed and therefore strong in these molecules, but spontaneous emission is forbidden. The absorption transactions occur between the molecules' delocalized n-orbitals from the 1¹A_{g} singlet ground state to the 1¹Bᵤ singlet excited state (see inset of FIG. 1), giving rise to broad absorption bands (˜100 nm width) in the blue and near-UV wavelength regions. The absorption bands shift to longer wavelengths with increasing number of effective conjugation length of the respective molecule. The absorption of the relatively short phytofluene molecule (five conjugated C═C bonds) is centered at ˜340 nm, and the much longer lycopene molecule (eleven C═C bonds) is centered at ˜450 nm, for example. All carotenoid molecules show a clearly resolved vibronic substructure due to weak electron-phonon coupling, with a frequency spacing of ˜1400 cm⁻¹.

In all carotenoids, any optical excitation within their absorption bands leads to only very weak luminescence signals. The associated extremely low quantum efficiency of the luminescence is caused by the existence of a second excited singlet state, a 2¹A_{g} state, which lies below the 1¹Bᵤ state (see FIG. 1 inset). Following excitation into the 1¹Bᵤ state, the carotenoid molecule relaxes very rapidly, within ˜200-250 fs, via nonradiative transitions, to this lower 2¹A_{g} state, from which electronic emission to the ground state is parity-forbidden (dashed, downward pointing arrows in inset of FIG. 1). The low 1¹Bᵤ→1¹A_{g} luminescence efficiency (10⁻⁵-10⁻⁴) and the absence of 2¹A_{g}→1¹A_{g} fluorescence of the molecules allows one to detect the resonance Raman scattering (RRS) response of the molecular vibrations (shown as solid, downward pointing arrow in inset of FIG. 1) without potentially masking fluorescence signals. Specifically, RRS detects the stretching vibrations of the polyene backbone as well as the methyl side groups. The carotenoid molecules' carbon-carbon single-bond and double-bond stretch frequencies each generate a spectrally sharp, resonantly enhanced Raman signal when the molecules are excited in any of their vibronic absorption transitions in the visible wavelength region. The RRS lines are shifted from the excitation light frequency by exactly the amount of the vibrational stretch frequency, i.e. by 1159 and 1525 cm⁻¹, respectively. Since these frequencies are relatively large, these offsets translate into large wavelength shifts of several ten nm when the carotenoid molecules are excited in the visible. Superimposed on a large skin fluorescence background due to other chromophores, the spectrally very narrow RRS lines are readily isolated from the excitation light and background fluorescence. A medium-resolution (<1 nm) spectrograph suffices, and their intensities can be easily quantified with a linear detector array of suitable high dynamic range. Choosing an excitation wavelength in the spectral vicinity of 480 nm, RRS measures the combined concentrations of all resonantly excited carotenoids in skin, including beta-carotene, lycopene, beta cryptoxanthin, lutein and zeaxanthin. Phytoene and phytofluene, two carotenoids found in skin that have shorter conjugation lengths and corresponding absorptions in the UV, are not detected under visible light excitation conditions.

In the human retina, RRS can be used to measure the combined concentration of lutein and zeaxanthin in the ˜1 mm diameter macular region. This can be achieved with spatially integrating [I. V. Ermakov, R. W. McClane, P. S. Bernstein, and W. Gellermann, “Resonant Raman detection of macular pigment levels in the living human retina,” Optics Letters 26, 202-204 (2001)] or with spatially resolved imaging configurations [M. Sharifzadeh, D.-Y. Zhou, P. S. Bernstein, and W. Gellermann, “Resonance Raman Imaging of Macular Pigment Distributions in the Human Retina,” Journal of the Optical Society of America, JOSA A 25, 947-957 (2008)]. One of the preferred body sites for RRS based skin carotenoid measurements has been the palm of the hand or heel of the foot because the dermal melanin pigment levels at these tissue sites are lighter and less variable among individuals of different racial and ethnic backgrounds. Additionally, the stratum corneum, the outer dermal tissue layer, is relatively thick in the palm or heel (at least ˜400 μm). This insures that the excitation light does not penetrate beyond this strongly scattering layer (light penetration depth ˜200 μm) into the deeper tissue layers, where it could excite other, potentially confounding chromophores. In field applications with portable instrument configurations, the suitability of the RRS methodology could be demonstrated for the rapid measurement of large subject populations. Measurements of the palms produced a bell-shaped distribution with significant width (˜50% of the central value), proving that important characteristics of an objective biomarker of carotenoid status, such as inter-subject variability, could be easily reproduced in a non-invasive fashion [I. V. Ermakov, M. R. Ermakova, R. W. McClane, and W. Gellermann, “Resonance Raman detection of carotenoid antioxidants in living human tissues,” Optics Letters 26, 1179-1181 (2001)].

Based on these initial results, RRS based skin carotenoid detection could be readily developed for commercial applications in the nutritional supplement industry. For field applications in this industry, a portable RRS instrument was developed, initially based on a low-power compact 473 nm solid state laser/65 mm focal length spectrograph/CCD detector combination [I. V. Ermakov, M. Sharifzadeh, M. R. Ermakova, and W. Gellermann, “Resonance Raman Detection of carotenoid antioxidants in living human tissue,” Journal of Biomedical Optics, 10, 064028, 1-18 (2005)]. In a later stage, a more rugged, non-laser version, was developed, based on spectrally narrowed LED excitation in combination with photomultiplier detection [S. D. Bergeson, J. B. Peatross, N. J. Eyring, J. F. Fralick, D. N. Stevenson, and S. B. Ferguson, “Resonance Raman measurements of carotenoids using light emitting diodes,” J. Biomed. Optics 13, 044026 (2008)]. Presently, about ten thousand portable RRS instruments are in use in the nutritional supplement industry, with the total number of measured subjects reaching more than 10 million. Importantly, the method proves the efficacy of carotenoid-containing nutritional supplement formulations in this field

The acceptance of RRS in the scientific and medical arena had to await a rigorous validation of this novel optical concept with biochemically (i.e., HPCL-) derived carotenoid levels. Initially it was shown that carotenoid levels measured with RRS in the inner palm of the hand correlate strongly and significantly with HPLC-derived carotenoid levels of fasting serum, thus validating the method in an indirect way [W. Gellermann, J. A. Zidichouski, C. R. Smidt, and P. S. Bernstein, “Raman detection of carotenoids in human tissue,” in Carotenoids and Retinoids: Molecular Aspects and Health Issues, L. Packer, K. Kraemer, U. Obermueller-Jervic, and H. Sies, Eds., Chapter 6, pp. 86-114, AOCS Press, Champain, Ill. (2005)]. More recently, direct validation experiments were completed that involved skin carotenoid RRS measurements followed by biopsy of the measured tissue volume and subsequent HPLC analysis [I. V. Ermakov and W. Gellermann “Validation model for Raman based skin carotenoid detection,” Archives of Biochemistry and Biophysics, 504, 40-9 (2010); S. T. Mayne, B. Cartmel, S. Scarmo, H. Lin, D. Leffel, E. Welch, I. V. Ermakov, P. Bohsale, P. S. Bernstein, and W. Gellermann, “Noninvasive assessment of dermal carotenoids as a biomarker of fruit and vegetable intake,” Am. J. Clin. Nutr. 92, 794-800 (2010)]. Again, a high correlation was found between both methods. Based on these validations, RRS is now finding increased use in Nutrition Science, where it provides insight, with high statistical significance, into the health effects of diets, detrimental effects of external stress factors, such as smoking, and general nutritional differences between distinct populations. In addition, the method is finding increased use as rapid objective biomarker for tissue antioxidant status in medical areas such as Cancer Prevention Research and Neonatology.

A further non-invasive optical method for the assessment of skin carotenoid levels is based on reflection spectroscopy. Particularly useful is a pressure-mediated version of reflection spectroscopy that allows one to assess skin carotenoid levels after temporal removal of interfering blood chromophores [I. V. Ermakov and W. Gellermann “Dermal carotenoid measurements via pressure-mediated reflection spectroscopy” J. Biophotonics 5, 55-570 (2012)]. This method is disclosed in U.S. Pat. Appl. Pub. No. 2009/0306521 A1, the disclosure of which is incorporated by reference herein. This reflection method holds promise as a particularly simple and inexpensive method since it does not require any narrow-band light sources for excitation, since it has significantly higher signal levels and since it therefore requires less complex instrumentation.

Basic reflection spectroscopy has been used previously for the quantification of carotenoids in the macular region of the human retina (“macular pigment”) [U.S. Publication No. 2007/0252950, Reflectometry Instrument and Method For Measuring Macular Pigment] and in skin [W. Stahl, U. Heinrich, H. Jungmann, J. von Laar, M. Schietzel, H. Sies, and H. Tronnier, “Increased dermal carotenoid levels assessed by noninvasive reflection spectrophotometry correlate with serum levels in women ingesting betatene,” J. Nutr. 128, 903 (1998); W. Stahl, U. Heinrich, H. Jungmann, H. Tronnier, and H. Sies, “Carotenoids in Human Skin: Noninvasive Measurement and Identification of Dermal Carotenoids and Carotenol Esters,” Methods in Enzymology 319, 494-502 (2000)]. In retinal reflection spectroscopy, the macular carotenoids (which in contrast to skin comprise only two carotenoid species, i.e. lutein and zeaxanthin), are derived from a double-path propagation of white light through all ocular layers from the cornea to the reflective sclera behind the retina, and back. The quantification of carotenoids is possible with the help of a multi-layer, sequential, straight-light-path transmission model, in which the individual absorption and/or scattering effects of all ocular layers are described with respective absorption and/or scattering coefficients. The retinal carotenoid levels, concentrated in the macula, are derived from a multi-parameter fit of the calculated reflection spectra to the measured spectra.

In human skin, the much stronger light scattering caused by the outer stratum corneum layer does not permit the assumption of tissue light propagation in and modeling of straight light paths. Furthermore, there is no effective internal interface that could be used as a reflector. Instead, it has been attempted to calculate carotenoid levels from first principles, taking into account the inhomogeneity of chromophore distributions in the living tissue in this earlier approach, and using a complex spectral de-convolution algorithm with multi-compartment modeling for skin chromophores. A significant correlation between baseline skin and serum carotenoid levels could be demonstrated in a 12-week β-carotene supplementation study. Also, an apparent rise of skin carotenoid levels could be demonstrated in response to supplementation in a small group of volunteer subjects [F. Niedorf, H. Jungmann, and M. Kietzmann, “Noninvasive reflection spectra provide quantitative information about the spatial distribution of skin chromophores,” Med. Phys. 32, 1297-1307 (2005)]. However, the interpretation of reflection spectra within the diffusive light transport model in turbid media was recognized to be problematic for the assessment of the relatively weakly absorbing carotenoid chromophores [F. Niedorf, H. Jungmann, and M. Kietzmann, “Noninvasive reflection spectra provide quantitative information about the spatial distribution of skin chromophores,” Med. Phys. 32, 1297-1307 (2005)], and the methodology has not found widespread application.

A further attempt to derive skin carotenoid concentrations has explored skin color saturation measurements [S. Alaluf, U. Heinrich, W. Stahl, H. Tronnier, and S. Wiseman, “Dietary Carotenoids Contribute to Normal Human Skin Color and UV Photosensitivity,” J. of Nutrition 132, 399-403 (2002)]. In this method, one of the color tri-stimulus values, the b*-value, was measured and compared to the chromaticity diagram of a white reflection standard. Since the b*-value measures the color saturation from the yellow to the blue region, it can be expected to be influenced by the absorption of skin carotenoids occurring in this spectral range. However, the measurements are influenced not only by the carotenoid absorption but also by the superimposed absorption and scattering effects of blood and melanin, thus leading to rather unspecific results.

Pressure-mediated reflection spectroscopy derives skin carotenoid levels empirically by comparing reflection derived carotenoid absorption levels with background absorption/scattering levels in tissue where confounding blood chromophores have been temporally squeezed out. The instrumentation uses simple, spectrally broad, light excitation. The light reflected from the skin surface is measured spectrally resolved with a spectrograph/CCD detector combination or, as an alternative, measured just at a few suitable discrete wavelengths within and outside the carotenoid absorption range, respectively. Pressure-mediated reflection spectroscopy has already been demonstrated to reliably track skin carotenoid level in subjects consuming carotenoid rich juices [I. V. Ermakov and W. Gellermann, “Dermal carotenoid measurements via pressure mediated reflection spectroscopy,” J. Biophotonics 5, 559-570 (2012)].

### Optical Properties of Hemoglobin in Biological Tissue

Hemoglobin, the iron-containing oxygen transport protein in red blood cells, absorbs strongly in the visible wavelength region. The oxygen-carrying variant, oxy-hemoglobin, features two partially resolved absorption bands with peaks at about 530 and 580 nm, respectively, whereas the oxygen-depleted variant, de-oxy-hemoglobin, has more of a single, broad-band absorption with peak at 560 nm. Care must be taken in tissue carotenoid measurements that the identifying absorption is not masked by the absorption bands of the hemoglobin chromophores. This can be achieved by judicious choice of the tissue site, a suitable light propagation scenario, and/or optimized choice of the detection wavelength. Preferably the latter should be outside the wavelength range of the blood chromophores.

### Absorption Spectroscopy

The RRS and reflection methodologies described above, measure carotenoid levels in biological tissue such as living skin only in the superficial tissue layers, down to a relatively shallow tissue depth of a fraction of a millimeter. This limitation is posed mainly by the strong light scattering in the stratum corneum layer, which causes high optical losses for any light in the visible wavelength region, including the excitation light, Raman scattered light or reflected light. In the present system and methods, we describe a new optical method that overcomes this drawback. Based on absorption spectroscopy, the new method is capable of measuring levels of tissue carotenoids and related biological compounds throughout the whole tissue thickness of a living body extremity or appendage of up to several cm. For the first time, this makes it possible to measure carotenoid levels in important living human body parts such as a hand, a finger, an ear lobe, a skin fold, or similar, and in this way to obtain a quantitative measure including tissue-internal compound levels rather than only surface concentrations. As a quantitative measure of the tissue compound concentration we choose the logarithmic ratio of the transmitted light intensity, Iₒᵤₜ, and a reference light intensity, I_{ref}, in the wavelength region of interest, and determine the carotenoid absorption of interest after subtraction of a scattering/absorption background that is due to other spectrally overlapping tissue chromophores. Specifically, we determine the optical density

O.D.=lg T⁻¹

from these measurements, where T is the percentage transmission of the input light through the sample, i.e. T=Iₒᵤₜ/I_{ref}. Measuring the absorption of the carotenoids over time, it is possible to track changes in concentration caused by dietary changes.

Comparing transmission-derived biological carotenoid levels with the disclosed method in tissues with different compositions, for example in tissue containing or not containing internal bone, respectively, it may be possible to obtain selective information of carotenoid concentrations in specific internal tissue components. For example, it may be possible with the disclosed method to determine carotenoid levels selectively in internal fat layers, in cartilage, or in bone.

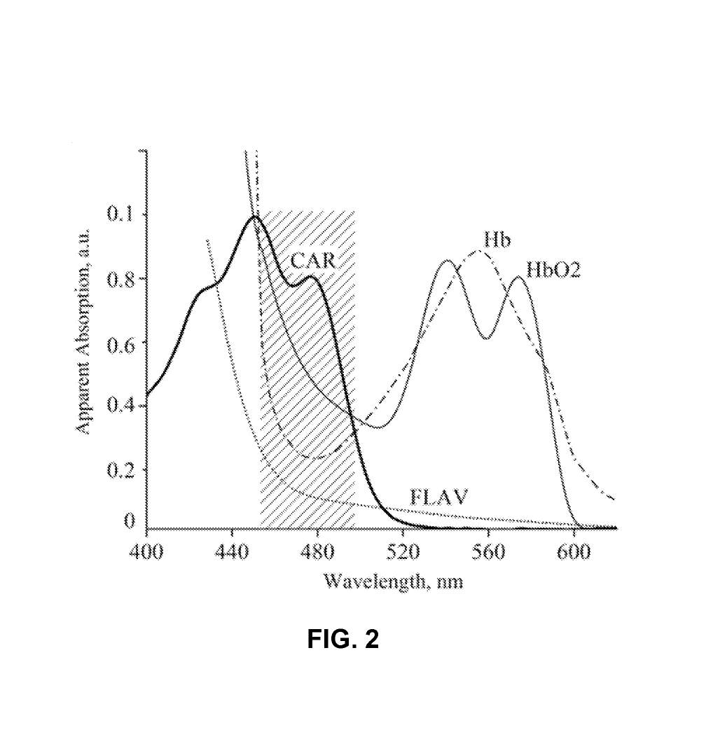

FIG. 2 illustrates model absorption spectra of the main absorbing chromophores in melanin-free human skin tissue. Specifically, FIG. 2 illustrates oxygenated hemoglobin, HbO2 (thin solid line), de-oxygenated hemoglobin, Hb (thin dash-dotted line), and carotenoids (CAR) (thick solid line). The illustrated carotenoid absorption is shown for a solution of beta-carotene. Also shown in FIG. 2 is the absorption tail of flavonoids, (FLAV, (thin dotted line) existing in skin in small concentrations. FIG. 2 suggests that a spectral window most useful for carotenoid detection has to be centered in the 460-500 nm spectral range (shown cross hatched) since absorption effects of the other chromophores are relatively minimal in this region.

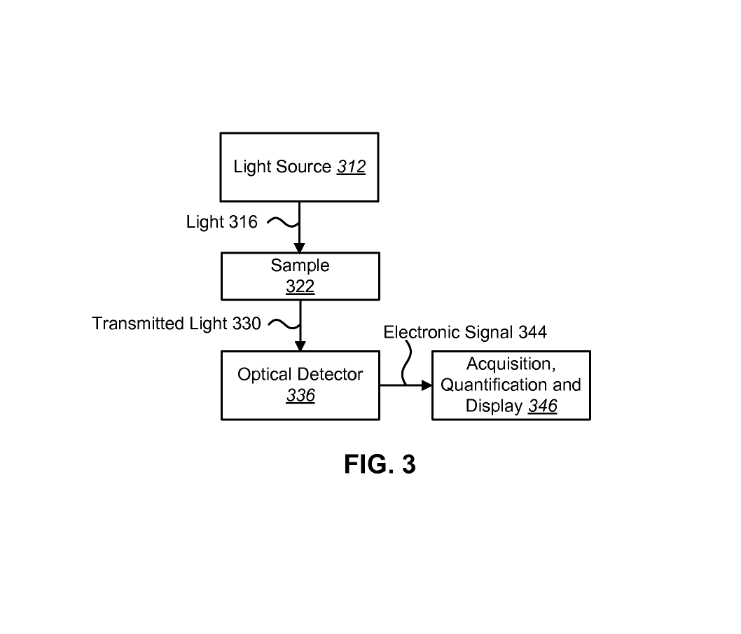

FIG. 3 is a block diagram illustrating one example of an absorption spectroscopy based apparatus for measuring and quantifying biological compounds in tissue. Biological compounds may include carotenoids, blood, water, etc. The biological compounds may be measured and quantified from living tissue samples and/or an excised tissue sample. The apparatus may include a light source 312, an optical detector 336, and data acquisition, processing, quantification and display module 346. The apparatus may quantify biological compounds found in a sample 322.

The light source 312 may illuminate light 316 on the sample 322. The light 316 may originate from a light emitting diode (LED) light source, a LED array, a conventional light source, and/or any other suitable broad band light source. For example, a low-cost LED light source may be used. One light source 312 or multiple light sources may be used. In some configurations, an optical fiber may be used to direct the light 316 generated by the light source 312.

The light source 312 may give off a spectrum of light 316 generated at wavelengths encompassing 480 nm, for instance from 400 nm to 600 nm. In other words, the light may be generated at wavelengths that may substantially overlap the absorption band of carotenoids. Additionally or alternatively, the light source 312 may give off light 316 generated at wavelengths encompassing 970 nm, for instance, from 800 nm to 1050 nm. In general, the light source 312 may give off a full spectrum of white light 316 that spans a variety of spectra.

The sample 322 may be a living tissue sample from a human, such as a finger, a skin fold, an earlobe, etc. The sample 322 may be a living tissue sample from another living organism. Alternatively, the sample 322 may be an excised tissue sample from a human, such as an excised piece of skin tissue or a bone sample, or an excised sample from a former living non-human organism. The sample can be much thicker than previously deemed possible for the absorption-based measurements of tissue carotenoids. For example, the sample 322 could range in thickness up to 3 cm. However, the sample 322 may be more or less thick. For example, the sample 322 may be a thin piece of excised skin tissue only a few mm thick or it may be a tissue fold such as the fold between thumb and index finger, or an ear lobe. Conversely, the sample 322 may be an animal bone that is several cm thick. The sample 422 should be thin enough to allow light 316 from the light source 312 pass through the sample 322 with sufficiently high transmitted light levels for rapid processing and calculation of absorption levels. In some configurations, a stronger light source 312 may be used to quantify and measure biological compounds from thicker samples 322.

The optical detector 336 may detect transmitted light 330 from the sample 322 in spectrally resolved detection configurations or at strategically chosen discrete wavelengths. For example, the optical detector 336 may measure the intensity of the light emitted from the sample 322. The optical detector 336 may include a spectrograph/charge coupled (CCD) or CMOS detector configuration, a photomultiplier tube, a photodiode detector and/or other optical detectors. In some configurations, the optical detector 336 may include a spatially integrating optical detector.

If the sample 322 is a human finger, the light source 312 may illuminate light 316 onto the finger. Light 316 may enter one side of the finger. The light 316 may pass through the finger. Transmitted light 330 may exit from an opposite side the finger. The transmitted might 330 may be detected at the optical detector 336.

The optical detector 336 may convert the detected light into an electronic signal. The optical detector may send the electronic signal 344 to the acquisition, quantification and display module 346.

The acquisition, quantification and display module 346 may analyze and quantify the electronic signal 344, and display a result using suitable data acquisition and processing routines. The result may include biological compound concentration levels.

Determining levels of biological compounds in the sample 322 may include processing the electronic signal 344 from the optical detector 336. Processing the electronic signal 344 may include analyzing and/or visually displaying the signal on a monitor (not shown) and/or other display. Processing the electronic signal from the optical detector 336 may further include converting the light signal into other digital and/or numerical formats. Data acquisition software may be used by the quantification and display module 346 to determine the levels of biological compounds in the sample 322.

For example, the quantification and display module 346 may analyze, quantify and display the levels of carotenoids, hemoglobin and/or water in the sample 322. Additionally, the quantification and display module 346 may compare concentration levels of carotenoids in the result to concentration levels of carotenoids in normal biological tissue to assess the risk or presence of a malignancy or other disease, or to track level changes in response to dietary supplementation.

Additionally, the quantification and display module 346 may assess the combined carotenoid and flavonoid antioxidant status of the living tissue or sample 322. In this way, the associated antioxidant status of the sample 322 may provide some indication of the level of fruits or vegetables consumed by a user from whom the sample 322 was taken or whose living tissue was measured. As one example, as a user increases his or her consumption of fruits and vegetables, his or her associated antioxidant status may positively change over time.

In some configurations, the quantification and display module 346 may be a computing device. The computing device may be a personal computer or may include other computing devices.

In some configurations, the quantification and display module 346 may be in electronic communication with the light source 312. For example, the quantification and display module 346 may compare transmitted light 330 in relation to the light 316 given off at the light source 312. Additionally, the light source 312 can provide input and receive feedback from the quantification and display module 346.

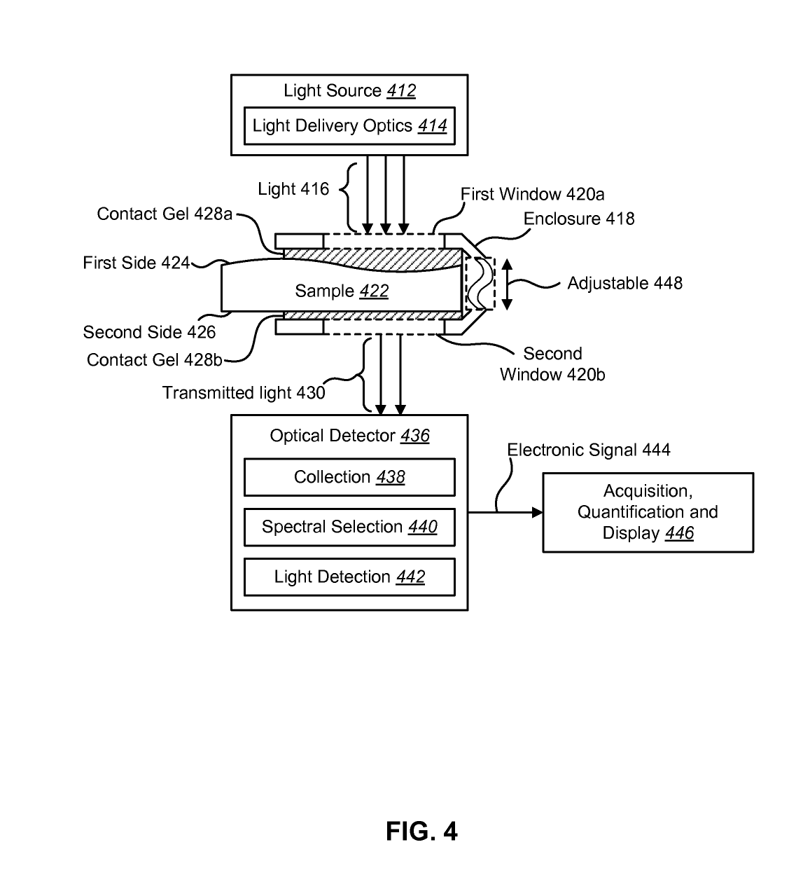

FIG. 4 is a block diagram illustrating a more detailed example of an absorption spectroscopy based apparatus for measuring and quantifying biological compounds in tissue. Biological compounds may include carotenoids levels, blood levels, water levels, etc. These biological compounds may be found in living tissue such as a finger, earlobe, skin folds, etc. Additionally, biological compounds may be found in excised tissue of suitable thickness.

The apparatus may include a light source 412, an enclosure 418, an optical detector 436 and an acquisition, quantification and display module 446. In some configurations, the components may be combined in a single apparatus. In other configurations, the components in the apparatus may be independent of each other. In other words, the components may form a system.

The light source 412 may include light delivery options 414 such as beam expanders, filters, apertures, shutters, etc. In one configuration, a beam expander and filter may be employed to enlarge and/or reduce the light to a predetermined size and/or shape on the sample 422. In other configurations, a beam expander and filter may expand and/or reduce the light to a sample 422 with other predetermined shapes and/or areas. For example, the beam expander and filter may expand and/or reduce the light to predetermined shapes such as an ellipse, an annulus, a polygon, multiple ellipses and/or other predetermined shapes. In another example, the beam expander and filter may expand and/or reduce the light to predetermine other excitation and detector areas.

The light source 412 may illuminate light 416 on the sample 422. The light 416 may be a light emitting diode (LED) light source, a LED array, a conventional tungsten light source and/or other light sources. For example, a low-cost LED light source may be used. One light source 412 or multiple light sources may be used. In some configurations, an optical fiber may be used to direct the light generated by the light source 412.

The light source 412 may give off a spectrum of light 416 generated at wavelengths encompassing 480 nm, for instance from 400 nm to 600 nm. In other words, the light may be generated at wavelengths that may substantially overlap the absorption band of carotenoids in living tissue or other organic samples. Additionally or alternatively, the light source 412 may give off light 416 generated at wavelengths encompassing 970 nm, for instance, from 800 nm to 1050 nm. In other words, the light may be generated at wavelengths that may substantially overlap the absorption band of tissue hydration. In general, the light source 412 may give off a full spectrum of white light 416 that spans a variety of biological compound absorption spectra.

The enclosure 418 may encompass the sample 422. The light source 412 may shine light 416 into the enclosure 418. The enclosure 418 may include an opening where the sample 422 may be inserted.

The enclosure 418 may have a first window 420a and a second window 420b. The first window 420a may allow light 416 from the light source 412 to enter into the enclosure 418. The second window 420b may allow transmitted light 430 from the sample to exit the enclosure 418. The enclosure 418 may otherwise prevent the light 416 and/or other stray light from exiting the enclosure 418 other than the transmitted light 430. For example, the enclosure 418 may be adjustable 448 to prevent stray light from exiting the enclosure 418. If light other than the transmitted light 430 exits the enclosure 418, an inaccurate result may occur.

A contact gel 428a, 428b may be employed to fill the space between the enclosure windows and tissue sample. Contact gel 428a, 428b may reduce light reflection from intermediate optical surfaces. In other words, the contact gel 428a, 428b may prevent airspace between the first window 420a and the sample 422, as well as between the second window 420b and the sample 422.

The sample 422 may be a living tissue sample from a human, such as a hand, a finger, a skin fold, an earlobe, a portion of the nose, etc. A human finger sample 422 may include skin, bone and fat. The area between the thumb and the index/pointer finger of the human hand may include two layers of skin. A human earlobe may include cartilage and no bone.

The sample 422 may be, for example, living breast tissue. This may be beneficial as carotenoids may have an impact on breast cancer. Current approaches for measuring biological compounds require sticking needles into the breast, sending light into the breast via fiber optics and measuring the light propagating between the fibers. Rather than using invasive approaches to measure biological compounds in human breast tissue, the present systems and methods described herein allow biological compounds to be measured using a non-invasive approach.

Additionally, the sample 422 may be an excised human tissue sample, such as an excised piece of skin tissue or a bone sample, or an excised sample from a former living organism. For example, a carrot slice or other vegetable may be used as the sample 422.

The sample 422 should be thin enough to allow light 416 from the light source 412 pass through the sample 422. In some configurations, a stronger light source 412 may be used to quantify and measure biological compounds from thicker samples 422.

The sample 422 may have a first side 424 and a second side 426. Light 416 from the light source 412 may illuminate the first side 424 of the sample 422. A portion of the light 416 may be absorbed by the sample 422 and a portion of the light 416 may be transmitted by the sample 422 as transmitted light 430. The transmitted light 430 may emerge from the second side 426 of the sample 422. The transmitted light 430 from the second side 426 of the sample 422 may pass through the second window 420b of the enclosure 418 and be captured by the optical detector 436.

In some configurations, there may be no gap between the light source 412 and the first window 420a of the enclosure 418. Additionally or alternatively, there may be no gap between the second window 420b of the enclosure 418 and the optical detector 436. In this manner, no stray light may interfere with the obtained results.

In another configuration, the light source 412 and/or the optical detector 436 may be part of the enclosure 418. For example, the light source 412 may be included in place of the first window 420a. Additionally or alternatively, the optical detector 436 may be included in place of the second window 420b. Adding the light source 412 and/or the optical detector 436 to the enclosure 418 may help to prevent stray light from interfering with any obtained results.

The optical detector 436 may detect transmitted light 430 from the second side 426 of the sample 422. The optical detector 336 may include a collection module 438, a spectral selection module 440 and a light detection module 442. The collection module 438 may collect the transmitted light 430. The collection module 438 may include a charge coupled device (CCD) camera, a CMOS detector, a photomultiplier tube, a photodiode detector and/or other optical detectors. A CCD array is an array of pixels that detects light intensities and wavelengths corresponding with the pixels. In some configurations, the collection module 438 may include a spatially integrating optical detector.

The spectral selection module 440 may filter out unwanted frequencies of collected light. For example, the spectral selection module 440 may filter out collected light outside of the 400 nm-600 nm wavelength. As another example, the spectral selection module 440 may filter out collected light outside of the band of hydrated tissues. In other words, the spectral selection module 440 may filter out signals from irrelevant or unwanted wavelengths.

Additionally or alternatively, the spectral selection module 440 may optionally include a spectrometer or spectrograph. For example, a spectrograph may be required to measure the carotenoid, hydration and/or hemoglobin levels in the sample 422. The spectrograph may be selected from commercial spectrograph systems such as a medium-resolution grating spectrograph that employs high light throughput and corresponding rapid detection with a compact, charge-coupled silicon detector array. For example, a spectrograph/CCD array light detection system can be used which employs a dispersion grating with 1200 lines/mm, and a one-dimensional, 1×2048, silicon CCD detector array, with 14×200 μm individual pixel area.

The light detection module 442 may detect the collected light. Additionally or alternatively, the light detection module 442 may convert the detected light into an electronic signal. The optical detector may send the electronic signal 444 to the quantification and display module 446. In some configurations, the light detection module 442 may be part of the spectrometer.

The acquisition, quantification and display module 446 may analyze and quantify the electronic signal 444 and display a result. The result may include biological compound concentration levels. Additionally or alternatively, the result may be a composite score based on the measured biological compounds in the sample 422.

Determining levels of biological compounds in the sample 422 may include processing the electronic signal 444 from the optical detector 436. Processing the electronic signal 444 may include analyzing and/or visually displaying the signal on a monitor (not shown) and/or other display. Processing the electronic signal 444 from the optical detector 436 may further include converting the light signal into other digital and/or numerical formats. Data acquisition software may be used by the quantification and display module 446 to determine the levels of biological compounds in the sample 422. For example, the quantification and display module 446 may analyze, quantify and display the levels of carotenoids, water, hemoglobin and/or other biological compounds in the sample 422.

The quantification and display module 446 may compare concentration levels of carotenoids and other biological compounds in the result to concentration levels of carotenoids and other compounds in normal biological tissue to assess the risk or presence of a malignancy or other disease. The quantification and display module 446 may assess the combined carotenoid and flavonoid antioxidant status of the sample 422. In this way, the associated antioxidant status of the sample 422 may provide an indication of the level of fruits or vegetables consumed by a user from whom the sample 422 was taken. As one example, as a user increases his or her consumption of fruits and vegetables, his or her antioxidant status may positively change over time.

In some configurations, the quantification and display module 446 may be a computing device. The computing device may be a personal computer or may include other computing devices. In some configurations, the quantification and display module 446 may be, in part, included on a mobile device (not shown). For example, the quantification and display module 446 may be part of an application located on a mobile deceive.

In some configurations, the quantification and display module 446 may be in electronic communication with the light source 412. For example, the quantification and display module 446 may compare transmitted light 430 in relation to the light 416 given off at the light source 412. Additionally, the light source 412 can provide input and receive feedback from the quantification and display module 446.

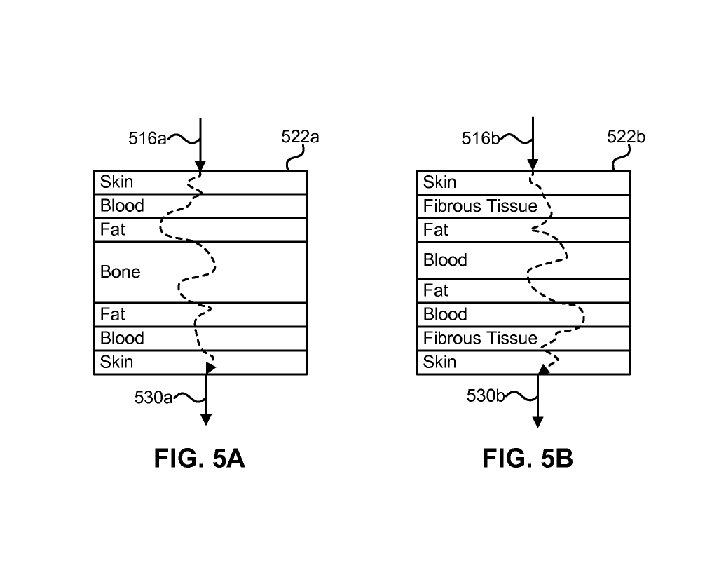

FIG. 5A illustrates light propagation through the various layers of tissue in a human finger. The sample 522a may include skin, blood, fat and bone. The skin layer may include the subcutaneous layers, epidermis and dermis. As one example, the sample 522a may be a human finger. Obtaining a composite score of carotenoids from a finger may provide representation of carotenoids levels in other parts of the body. Further, a composite carotenoid score obtained from light absorption provides more accurate results than a composite score obtained from measurement of reflected light from a superficial layer of skin.

In the case of a finger or another sample 522a that includes skin, blood, fat and bone, the light 516a from a light source 312 may have to pass through two layers of skin, blood and fat. The light 516a may be scattered and absorbed as it travels though the sample 522a. This scattering and absorption is illustrated as a dashed line. A portion of the incident light may exit as transmitted light 530a to be quantified and displayed.

Skin color is generally defined by the combined optical effects of melanin, blood, carotenoids and light scattering. Carotenoids are yellow in nature so they absorb blue light. Blood in the skin, on the other hand, does not strongly absorb blue light. Thus, blood has a reduced absorption effect on measuring carotenoid concentration in the blue wavelength region.

FIG. 5B illustrates light propagation through the various layers of tissue in a boneless body part, such as an ear lobe or a tissue fold. The bone-less sample 522b may represent skin, fibrous tissue, fat and blood. For example, the sample 522b may be a human earlobe. In this example, the sample 522b may include multiple layers of skin, fibrous tissue, blood and fat. The light 516b may be internally scattered, reflected and absorbed as it travels though the sample 522b, resulting in a path that severely deviates from a straight-light-path propagation inside the tissue. Illustrated as a dashed line inside the tissue, the transmitted light 530b may then exit the sample 522b to be quantified and displayed.

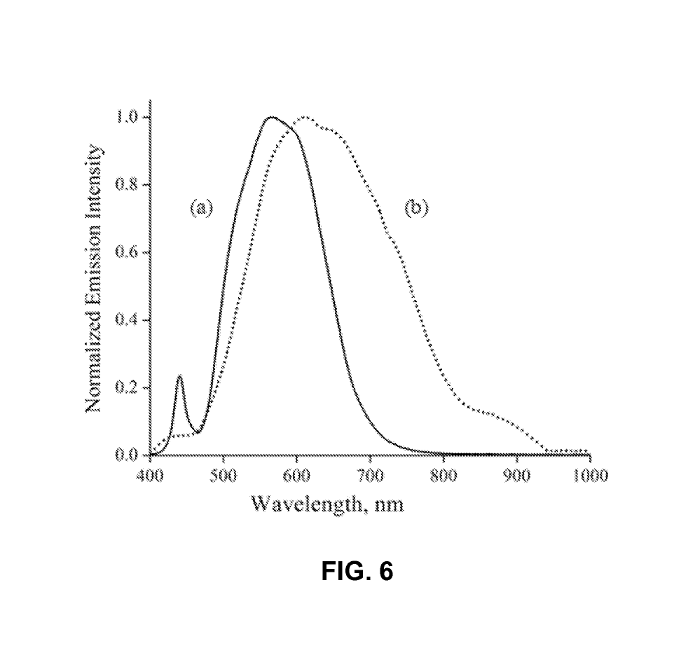

FIG. 6 illustrates typical emission spectra of two different light sources suitable for absorption measurements. For example, a white-light emitting diode, LED, (solid line (a), or light from a tungsten-halogen lamp (dotted line b) may be used to measure absorption levels of biological compounds in samples 322. Both sources of light may cover a wide spectral range from the near ultraviolet (UV) to the near infrared (IR) spectral range. The tungsten-halogen light source (b) may be better for biological compounds that are measured toward the near IR range. For example, using a tungsten-halogen source as a light source may be more effective at measuring water levels in a sample than using a white-LED. This is because water levels are generally measured at a wavelength of 970 nm.

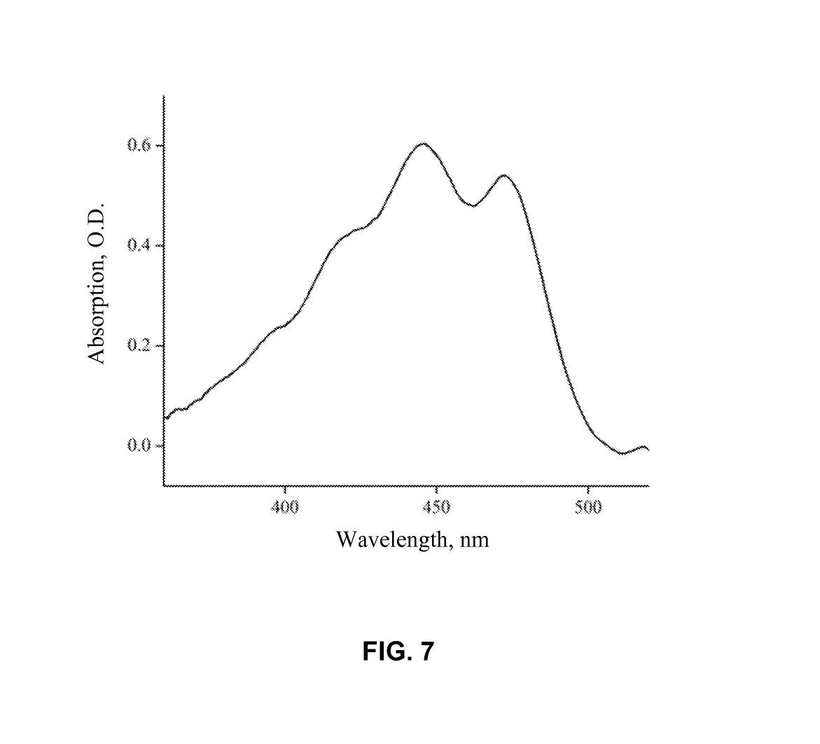

FIG. 7 illustrates the absorption spectrum of a 1 mm thick quartz cuvette filled with a methanolic carotenoid solution used as a sample 322. In this case, a tungsten halogen lamp was used as a light source 312. The carotenoid solution shows characteristic vibronic peaks in carotenoid absorption levels at 450 nm and 480 nm.

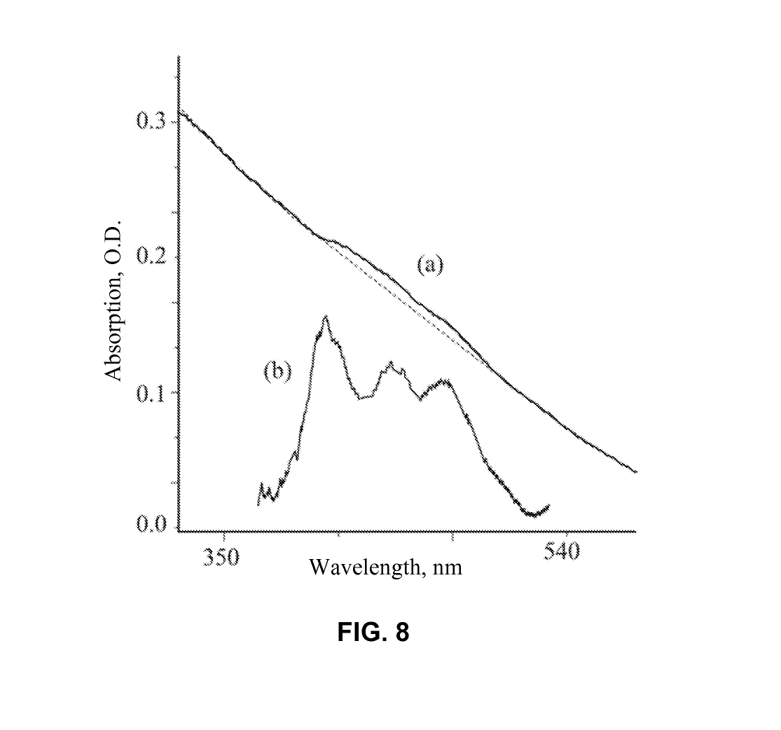

FIG. 8 illustrates transmission derived carotenoid absorption spectra of an excised, bloodless heel skin tissue sample 322 in the 350-540 nm wavelength range (trace (a), solid line). A dotted line represents the baseline to the spectrum. After subtraction of the baseline, the carotenoid absorption spectrum may be derived. This is shown with an expanded scale as insert (trace b). Importantly, each characteristic vibronic substructure feature of the carotenoid absorption within the tissue sample can be distinguished, thus clearly revealing the presence of carotenoids, and therefore allowing one to quantify their levels based on the strength of the absorption. An important aspect in these transmission-based tissue carotenoid measurements is a judicious choice of the input light intensity. It needs to be kept sufficiently low to transmit only diffusively scattered light to the detector on the opposite tissue side. In this way the internal tissue chromophores can impart their full absorption onto the spectral characteristics of the incoming light. Otherwise, if the light intensity is too high, the detector might see only ballistic transmitted photons, and the transmitted light spectrum would be unchanged relative to the input spectrum.

A possible method that may be used for the baseline estimation is a modified version of an algorithm termed “Signal Removal Methods (SRM),” as described by Schulze et al. [G. Schulze, A. Jirasek, M. M. L. Yu, A. Lim, R. F. B. Turner, and M. W. Blades, “Investigation of selected baseline removal techniques as candidates for automated implementation,” Appl. Spectrosc. 59, 545-574 (2005)].

SRM estimates a baseline using a smoothing routine or low-order polynomial fit to the entire measured spectrum. After the initial estimation of the baseline, those points in the spectrum that have higher intensities than the baseline will be stripped from the spectrum by replacing them with the value of the estimated baseline. After stripping, a new baseline estimate is generated, and this procedure is iterated until the new baseline estimate does no longer change or changes just a little between two consecutive iterations. Usually, this procedure is fast and ideal for automation.

The modified algorithm may employ the following steps. First, acquire a real spectrum with the spectrograph. Second, use a smoothing routine (e.g., Savitsky-Golay filtering) or low-order polynomial fitting through the original data points to generate a first-estimate baseline. Third, establish a threshold using the initial estimate to separate the signal from the baseline. The signal is the data above the threshold, and the baseline is the data below the threshold. Fourth, modify the original data by replacing any point valued higher than the threshold with the value of the threshold at that point (in other words, remove the signal). Fifth, apply Savitsky-Golay filtering or similar to the modified data set to provide a second estimate of the baseline. Sixth, repeat the signal removal step using the new threshold obtained with the Savitsky-Golay filter routine and apply Savitsky-Golay filtering again to the modified data set. Seventh, repeat the process until the iteration can be stopped due to reaching an iteration criterion or due to reaching a fixed number of iteration steps. Finally, subtract the best estimate baseline from the original spectrum to produce a baseline-subtracted spectrum.

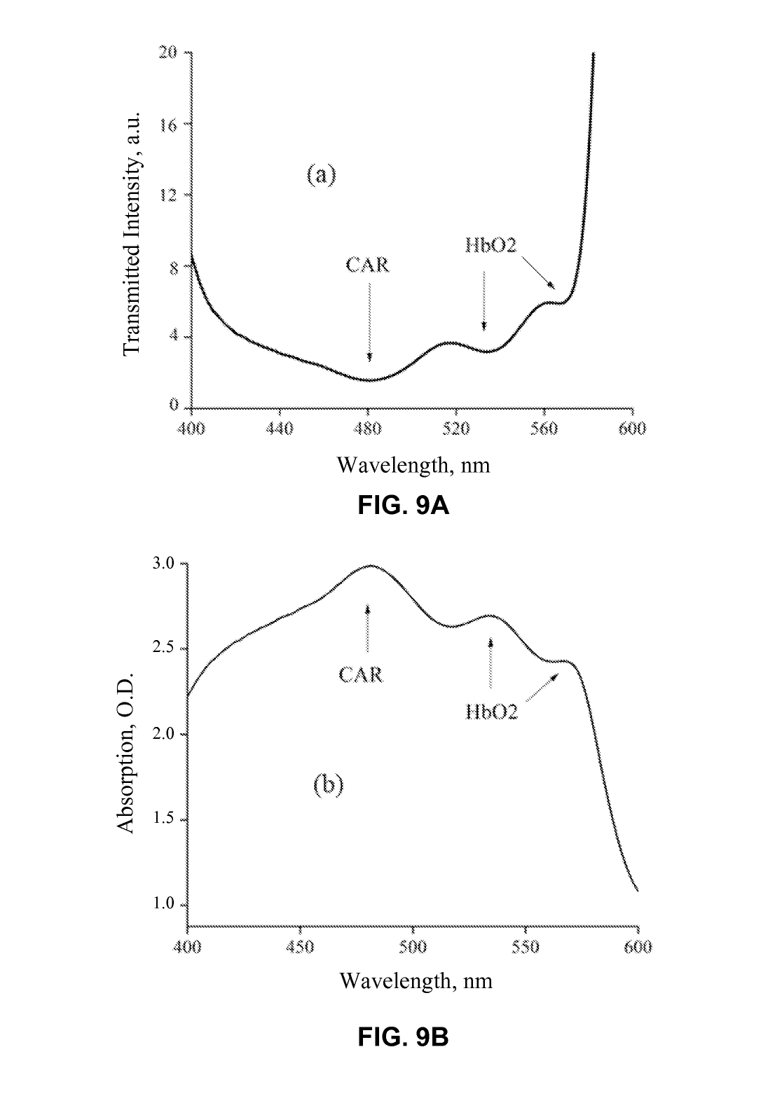

FIG. 9A illustrates the transmission spectrum measured for a living human finger as sample 322. The light 316 is from a white light source 312 and is measured with a spectrograph/CCD array instrument configuration. Because light 316 passes through a sample 322, only light 316 that is not absorbed or internally reflected exits the sample as transmitted light 330. The sample absorption at any wavelength can be estimate from the measured transmission at the corresponding wavelength by taking the logarithmic ratio between excitation light intensity and transmitted intensity. Additionally, because a strong signal may be emitted from the sample 322, a low-cost light source 312 may be used in obtaining transmission measurements.

FIG. 9 B illustrates the absorption spectrum derived from the transmission spectrum in FIG. 9 A. FIG. 9B reveals characteristic carotenoid and blood absorption features in the 400-600 nm wavelength range. For example, carotenoids (CAR) are identified through their characteristic absorption peak around 480 nm, and blood chromophore features, such as hemoglobin (HbO2), through their peaks at 530 and 580 nm.

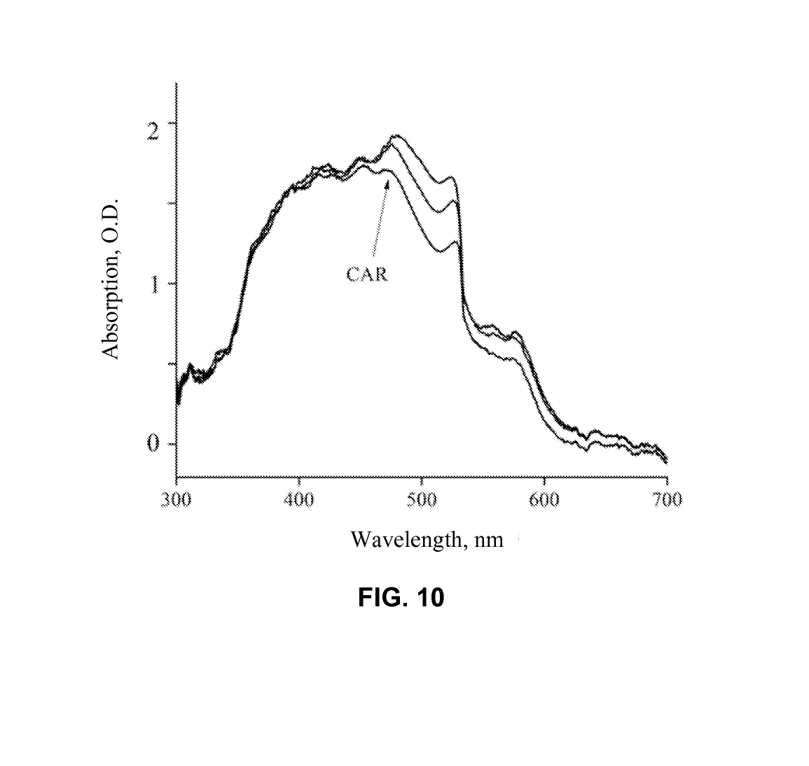

FIG. 10 illustrates the transmission-derived absorption spectra of different fingers (thumb, index and small finger) of a subject. Each solid line represents a different finger 322 on the same hand. FIG. 10 reveals different total amounts of carotenoids (CAR) in different fingers for the same subject. Note that the background absorption levels are similar outside the absorption region of carotenoids.

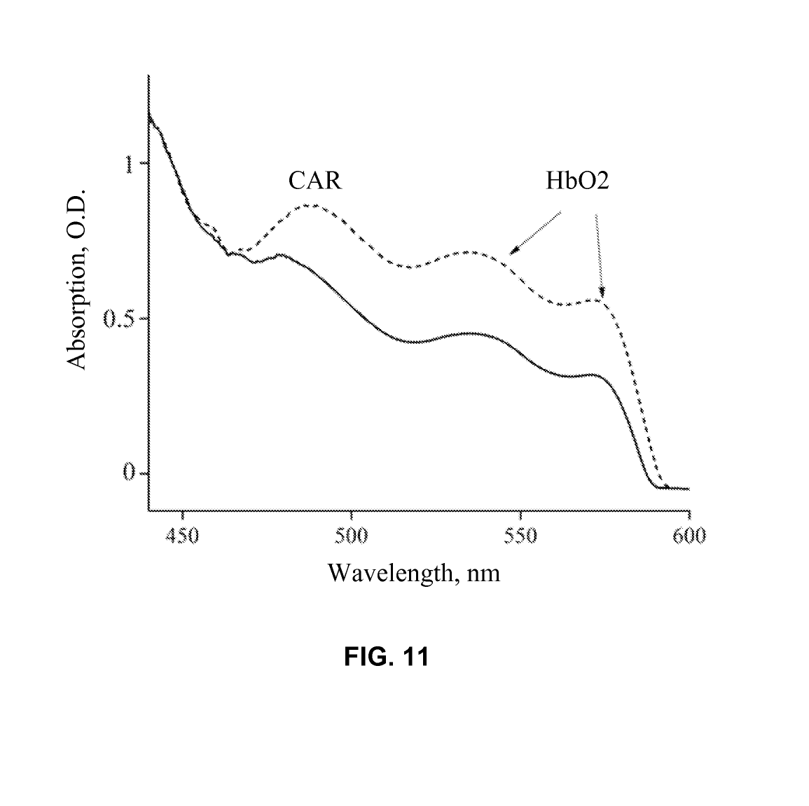

FIG. 11 illustrates absorption spectra of index fingers of comparable thickness from two different subjects. The solid line represents the index finger, used as a sample 322, from a first subject. The dashed line represents the index finger from a second subject. In both cases, a light emitting diode (LED) was used as the light source 312. FIG. 11 illustrates the difference in tissue carotenoid levels between the two subjects (higher for the dashed line spectrum compared to the solid line spectrum).

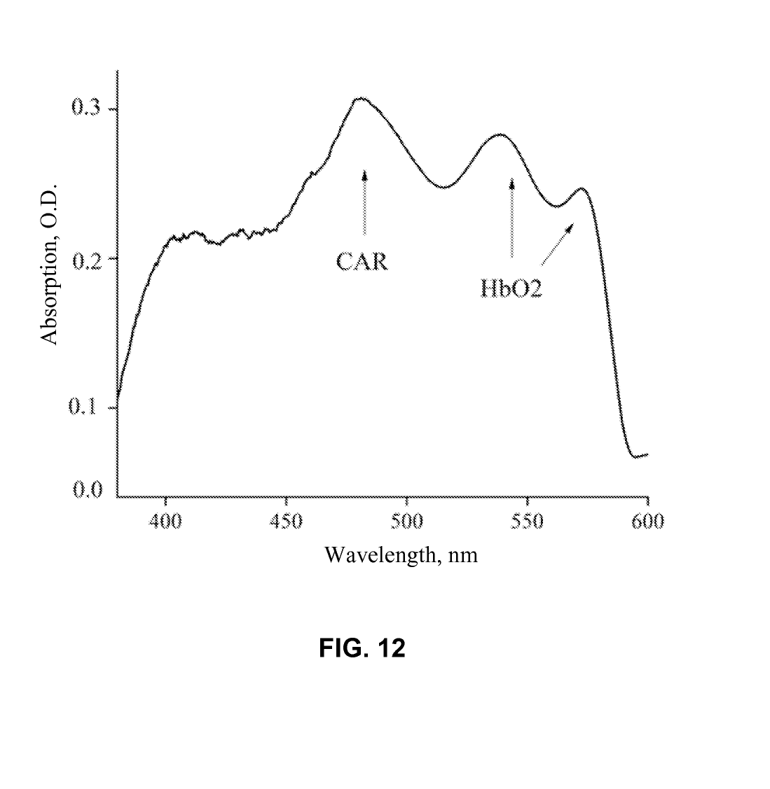

FIG. 12 illustrates the absorption spectrum of the thenar skin fold between thumb and index finger of a human subject. Measuring a thenar skin fold may increase measurement accuracy in quantifying biological skin carotenoid levels. This is because the thenar skin fold allows one to measure twice the skin carotenoid concentration due to the two skin surfaces. In other words, because the thenar skin fold presents the skin layer twice, and since in addition it features relatively low amount of blood and fat, the characteristic absorption features of biological compounds such as carotenoids occur twice for a tissue of interest, such as skin. This may result in a more accurate measurement of biological compounds in skin compared to optical methods that estimate skin levels from surface measurements.

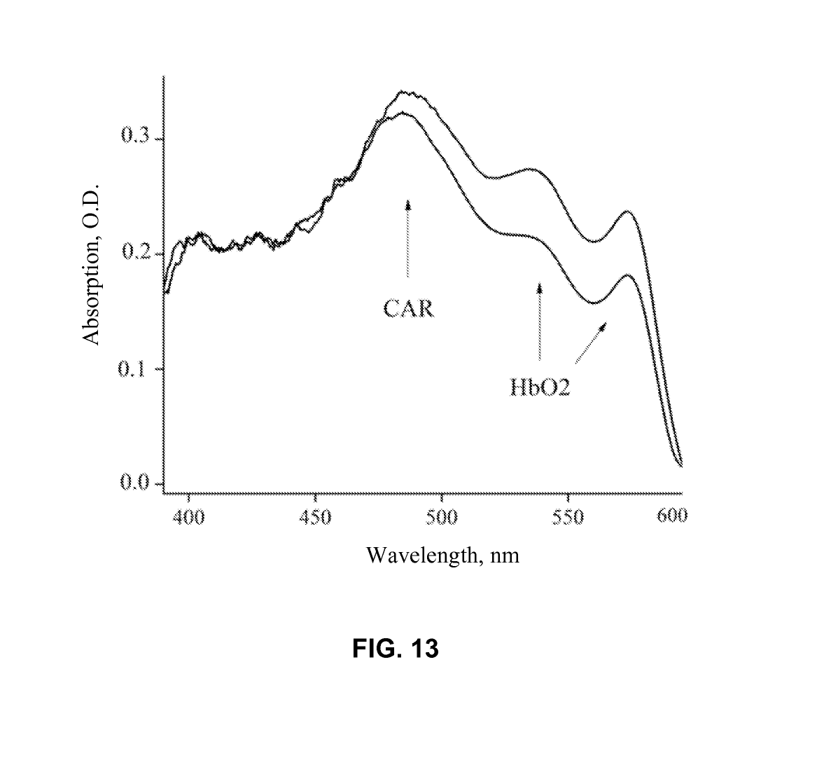

FIG. 13 illustrates absorption spectra of ear lobes of comparable thickness of two different subjects. Different carotenoid absorption strengths indicate different tissue carotenoid levels in the respective subjects. Measuring an ear lobe may provide an accurate measurement of carotenoids in internal tissue layers such as connective tissue because the ear lobe has only relatively thin outer skin layers.

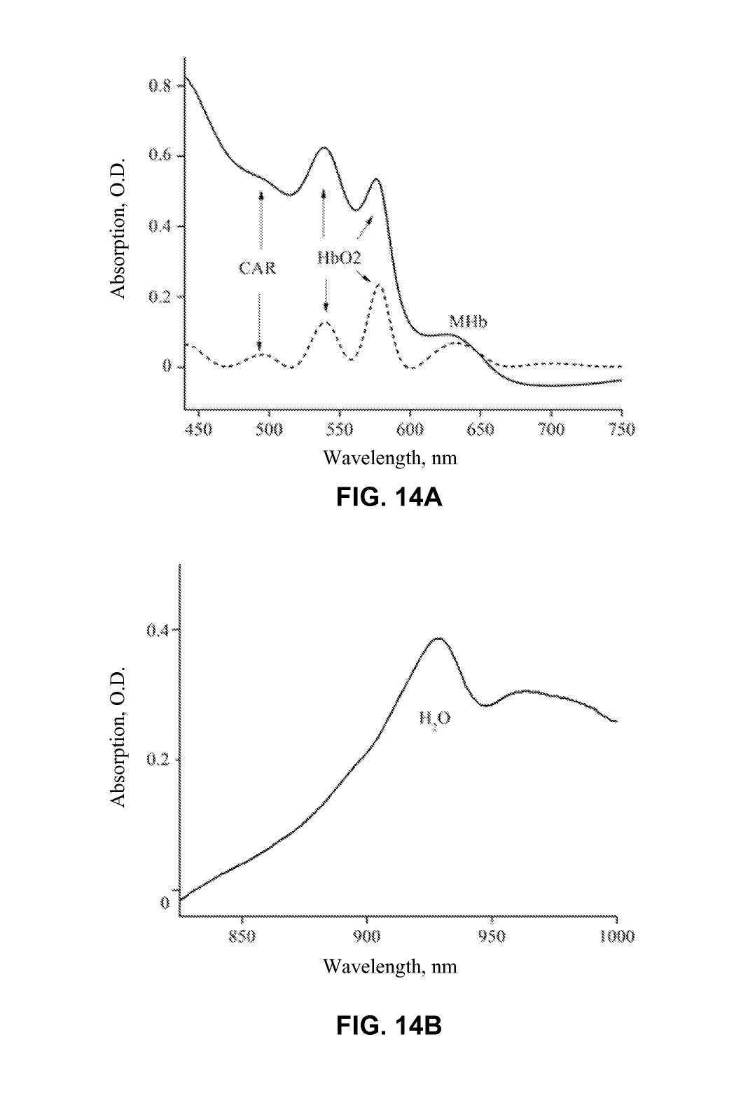

FIG. 14A and FIG. 14B illustrate absorption spectra from an excised rib tissue sample of a grass-fed cow as sample 322. The excised rib had all flesh and fat removed so that only the bone remained. Specifically, FIG. 14A illustrates both raw data (solid curve) and background-corrected data (dashed curve). These absorption results demonstrate the presence of carotenoids (CAR), hemoglobin (HbO2) and methemoglobin (MHb) in the grass fed cow rib, as evidenced by their respective characteristic wavelength positions in the absorption spectra. FIG. 14B illustrates the water content in the same bone tissue sample 322, showing the presence of small water content in bone material. Similar results were obtained from fat samples of grass-fed cows. Cows that were not grass-fed did not include any measurable carotenoid absorption. In this manner, the systems and methods described herein may provide a quick non-invasive approach for inspection of “organic” meat verses “non-organic” meat. In other words, organic meat from grass fed cows includes carotenoids while non-organic meat does not.

Human bone samples may also be measured using the systems and methods described herein. There is a high correlation of carotenoid levels between skin and bone in humans. In this manner, a user may measure and project bone health based on carotenoid levels measured in skin.

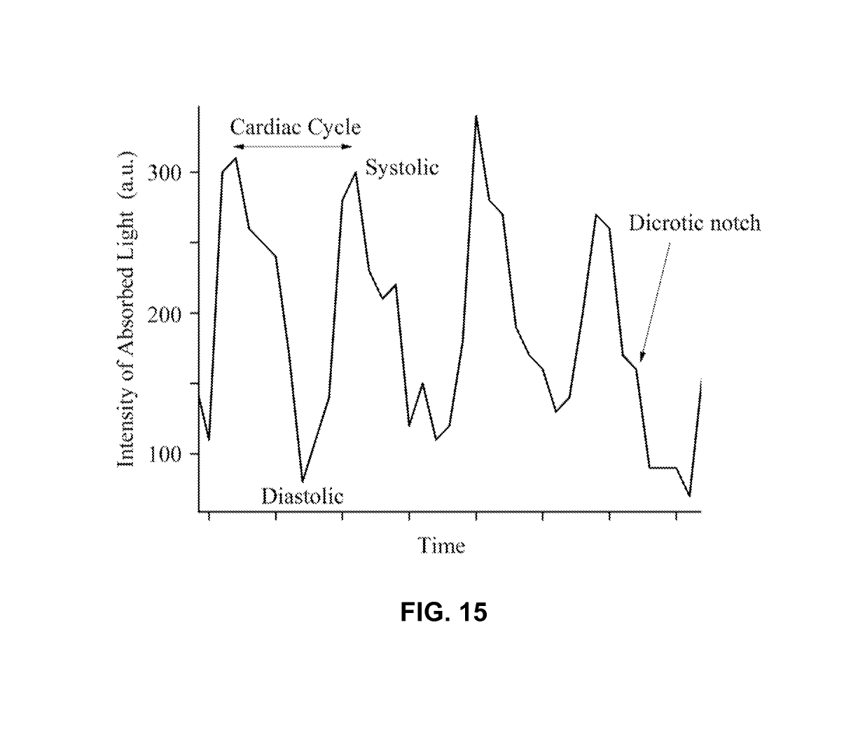

FIG. 15 illustrates time-resolved absorption behavior of a human finger in the carotenoid absorption region, showing a modulation of the carotenoid absorption due to the heart rhythm. FIG. 15 shows a time-resolved measurement of carotenoid absorption in the 480 nm region for a healthy human finger. A repetitive, alternating, carotenoid absorption component is evident, that is superimposed on a large, constant, carotenoid absorption background (the latter is not show in FIG. 15). The alternating carotenoid absorption has the same well-known temporal behavior as the absorption of blood when measured with standard pulse oximetry techniques [S. Palreddy, “Signal Processing Algorithms,” in: Design of Pulse Oximeters, J. G. Webster (ed.), Institute of Physics Publishing, Bristol, UK, and Philadelphia, USA (1997), chapter 9, pp. 124-158; P. D. Mannheimer, “The light interaction of pulse oximetry.” Anesth. Analg. 105 (6) S10-7 (2007)]. The effect is caused by absorption path changes for the light due to tissue-internal blood vessels expanding and contracting in rhythm with the human heartbeat. Since the carotenoid absorption changes with the same rhythm, the alternating carotenoid absorption must be caused by carotenoids circulating in the blood stream. Therefore, measuring and processing the time-resolved absorption behavior of carotenoid levels in living tissue as well as their static tissue absorption, it will be possible to separately determine carotenoid levels circulating in blood and carotenoid levels in tissue.

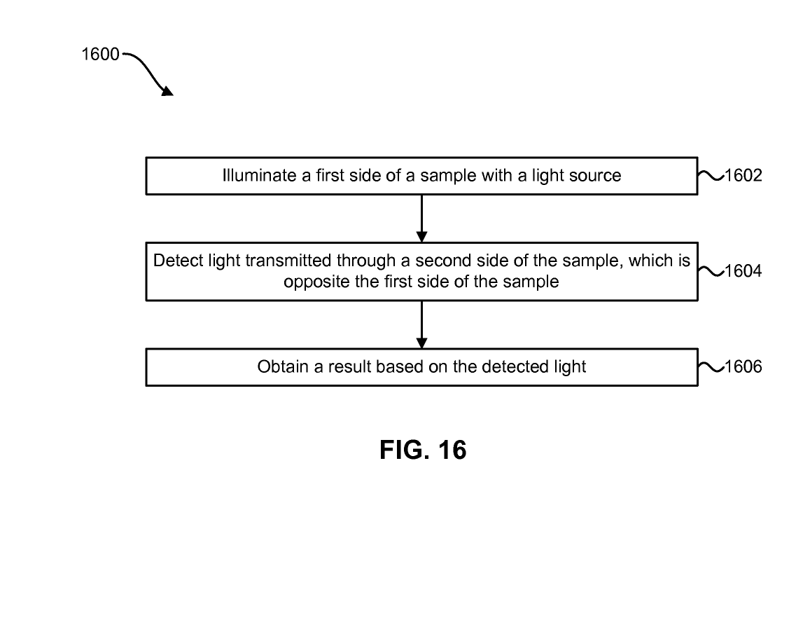

FIG. 16 illustrates a flow diagram of a method 1600 for measuring and quantifying biological compounds in tissue. The method 1600 includes illuminating 1602 a first side 424 of a sample 422 (e.g., tissue) with a light source 412. The light 416 from the light source 412 may be a light emitting diode (LED) light source, a LED array, a conventional light source, and/or other light sources. The light source 412 may be passed through one or more optical components.

Light 430 transmitted through a second side 426 of the sample 422 may be detected 1604. The second side 426 of the sample 422 may be opposite first side 424 of a sample 422.

The light 416 may be scattered, reflected and absorbed as it travels through the sample 422. A portion of the light 416 may be transmitted by the sample 422 as transmitted light 430.

Detecting 1604 transmitted light 430 may include measuring the spectrally resolved intensity of the light emerging from the sample 422 or the detection of light at strategically chosen discrete wavelengths. The transmitted light 430 may be detected 1604 by an optical detector 436 such as a CCD camera, a CMOS array, a photomultiplier tube, a photodiode detector and/or other optical detector. Detecting 1604 the transmitted light 430 may include converting the detected light into an electronic signal 444.

A result may be obtained 1606 based on the detected light. Obtaining 1606 the result may include processing the electronic signal 444 from an optical detector 436. Processing the electronic signal 444 may include analyzing and/or visually displaying the signal on a monitor and/or other display. Processing the electronic signal 444 may further include converting the light signal into other digital and/or numerical formats. Data acquisition software may be used by the computing device to determine the levels of biological compounds in the sample 422.

The biological compound levels may be compared to correlative data indicative of one or more pathologies or symptoms. Based upon the comparison, the presence, absence, or degree of one or more pathologies or symptoms may be determined.

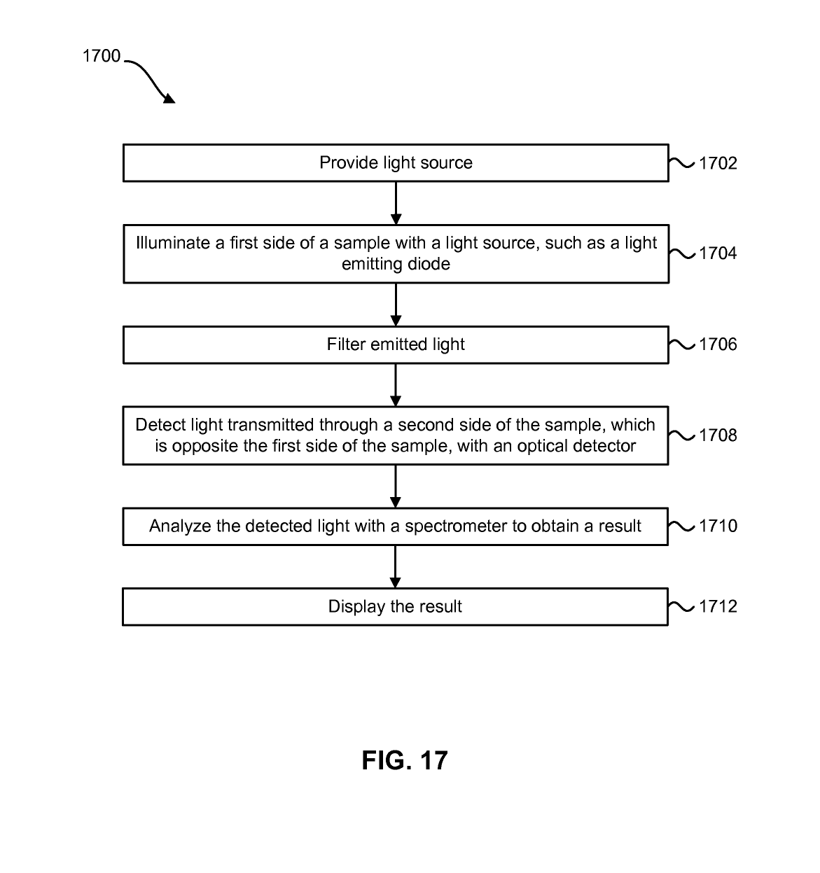

FIG. 17 illustrates a more detailed flow diagram of a method 1700 for measuring and quantifying biological compounds in a tissue. The method 1700 may include providing 1702 a light source 412. The light 416 may be generated at a wavelength that substantially overlaps the absorption band of carotenoids in the tissue.

In some configurations, the light source 412 may be filtered to allow/prevent certain wavelengths of light from reaching the sample 422. Filtering the light 416 generated by the light source 412 may include providing a narrow band pass filter, a laser line filter and/or other optical filters. Filtering the light 416 generated by the light source 412 may include filtering the light to generally exclude light with wavelengths outside a desired band. For example, the light 416 may be filtered to only include wavelengths that are typically absorbed by tissue chromophores.

The light source 412 may illuminate 1704 a first side 424 of a sample 422 with a light source 412. The light 416 from the light source 412 may be a light emitting diode (LED) light source, a LED array, a conventional light source and/or other light sources. The light source 412 may be passed through one or more optical components. The light source 412 may be directed towards the first side 424 of the sample 422. Directing the light source 412 to the first side 424 of the sample 422 may be accomplished using various optical elements and may include conditioning the light to create a target. For example, a lens may be used to expand the light to create about a 1 cm disk-shaped target. In other configurations, the light source 412 may be expanded and/or reduced to a target with other predetermined shapes and/or areas.

The light 416 transmitted through the sample 422 may be filtered 1706. Filtering 1706 the transmitted light 430 may include filtering out all unwanted spectra of light. For example, filtering 1706 may include using a long pass filter that filters light at 480 nm, 530 nm, 970 nm and/or other wavelengths.

The transmitted light 430 through a second side 426 of the sample 422 may be detected 1708. The second side 426 of the sample 422 may be opposite first side 424 of a sample 422. The transmitted light 430 may be detected 1708 by a photodiode detector, a photomultiplier tube, a CCD camera, a CMOS array, and/or other optical detectors. Detecting 1708 the transmitted light 430 may include converting the detected light into an electronic signal 444.

The detected light may be analyzed 1710 with a spectrograph/detector combination to obtain a result. Analyzing 1710 the electronic signal 444 may include processing the electronic signal 444 from an optical detector 436. Processing the electronic signal 444 may further include converting the light signal into other digital and/or numerical formats. Data acquisition software may be used by the computing device to determine the levels of biological compounds in the sample 422.

In one configuration, one measurement of the biological compound levels in the sample 422 may be made. In other configurations, multiple measurements may be taken. In configurations where multiple measurements of biological compound levels may be taken, the multiple measurements may be averaged to determine an average biological compound level for the subject. In some configurations where the biological compound levels may be averaged, the measurements may be taken from the same location on a user. For example, light used for each measurement may be directed to the same sample 422 location, such as an ear lobe. In other configurations, measurements may be taken from the different locations on the user's body. For example, samples 422 could be taken from a finger, a thenar skin fold and an ear lobe. In further configurations, a combination of measurements from the same and/or different locations may be used to determine the average biological compound levels in a user. The average biological compound levels may form a composite score/result.

The results may be displayed 1712. Displaying 1712 the result may include visually displaying the signal on a monitor and/or other display. For example, the display may be a mobile device such as a tablet computer or smartphone.

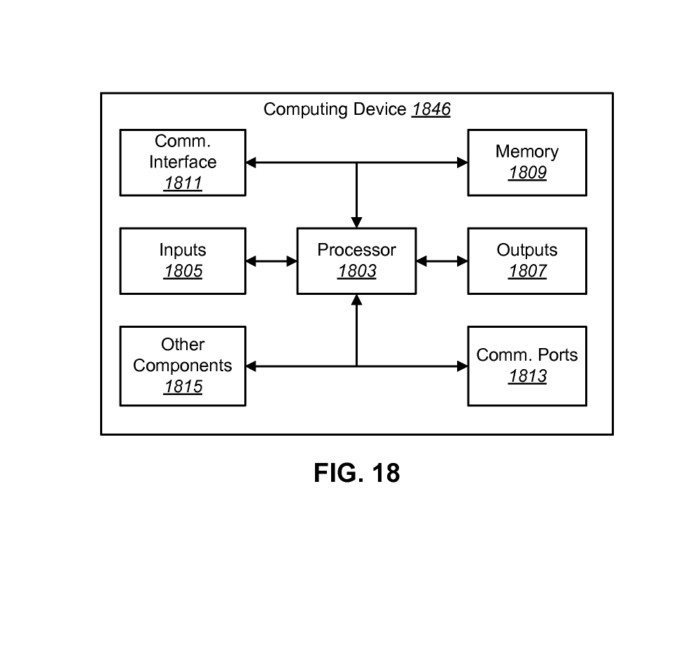

FIG. 18 is a block diagram illustrating various hardware components that may be used in a computing device 1846. The computing device 1846 may be one example of the quantification and display module 346. A computing device 1846 typically includes a processor 1803 in electronic communication with input components or devices 1805 and/or output components or devices 1807. The processor 1803 may be operably connected to input devices 1805 and/or output devices 1807 capable of electronic communication with the processor 1803, or, in other words, to devices capable of input and/or output in the form of an electrical signal. Example of devices 1846 may include the input devices 1805, output devices 1807 and the processor 1803 within the same physical structure or in separate housings or structures.

The computing device 1846 may also include memory 1809. The memory 1809 may be a separate component from the processor 1803, or it may be on-board memory 1809 included in the same part as the processor 1803. For example, microcontrollers often include a certain amount of on-board memory. The memory 1809 may store information such as lipofuscin levels and/or other information that may be used with the present systems and methods.

The processor 1803 may also be in electronic communication with a communication interface 1811. The communication interface 1811 may be used for communications with other devices 1846. For example, the communication interface 1811 may be used to communicate with the light source 312 and/or the optical detectors 336. Thus, the communication interfaces 1811 of the various devices 1846 may be designed to communicate with each other to send signals or messages between computing devices 1846.

The computing device 1846 may also include other communication ports 1813. In addition, other components 1815 may also be included in the computing device 1846.

Many kinds of different devices may be used with examples herein. The computing device 1846 may be a one-chip computer, such as a microcontroller, a one-board type of computer, such as a controller, a typical desktop computer, such as an IBM-PC compatible computer, a Personal Digital Assistant (PDA), a Unix-based workstation, a smart phone, etc. Accordingly, the block diagram of FIG. 18 is only meant to illustrate typical components of a computing device 1846 and is not meant to limit the scope of examples disclosed herein.

Information and signals may be represented using any of a variety of different technologies and techniques. For example, data, instructions, commands, information, signals, bits, symbols and chips that may be referenced throughout the above description may be represented by voltages, currents, electromagnetic waves, magnetic fields or particles, optical fields or particles or any combination thereof.

The various illustrative logical blocks, modules, circuits and algorithm steps described in connection with the examples disclosed herein may be implemented as electronic hardware, computer software or combinations of both. To illustrate this interchangeability of hardware and software, various illustrative components, blocks, modules, circuits and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The various illustrative logical blocks, modules, and circuits described in connection with the examples disclosed herein may be implemented or performed with a general purpose processor, a digital signal processor (DSP), an application specific integrated circuit (ASIC), a field programmable gate array signal (FPGA) or other programmable logic device, discrete gate or transistor logic, discrete hardware components or any combination thereof designed to perform the functions described herein. A general-purpose processor may be a microprocessor, but in the alternative, the processor may be any conventional processor, controller, microcontroller or state machine. A processor may also be implemented as a combination of computing devices, e.g., a combination of a DSP and a microprocessor, a plurality of microprocessors, one or more microprocessors in conjunction with a DSP core or any other such configuration.

The steps of a method or algorithm described in connection with the examples disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

The word “exemplary” is used exclusively herein to mean “serving as an example, instance, or illustration.” Any example described herein as “exemplary” is not necessarily to be construed as preferred or advantageous over other examples.

Some features of the examples disclosed herein may be implemented as computer software, electronic hardware or combinations of both. To clearly illustrate this interchangeability of hardware and software, various components may be described generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

Where the described functionality is implemented as computer software, such software may include any type of computer instruction or computer executable code located within a memory device and/or transmitted as electronic signals over a system bus or network. Software that implements the functionality associated with components described herein may comprise a single instruction, or many instructions, and may be distributed over several different code segments, among different programs, and across several memory devices.

The term “determining” (and grammatical variants thereof) is used in an extremely broad sense. The term “determining” encompasses a wide variety of actions and therefore “determining” can include calculating, computing, processing, deriving, investigating, looking up (e.g., looking up in a table, a database or another data structure), ascertaining and the like. In addition, “determining” can include receiving (e.g., receiving information), accessing (e.g., accessing data in a memory) and the like. In addition, “determining” can include resolving, selecting, choosing, establishing and the like.

The phrase “based on” does not mean, “based only on,” unless expressly specified otherwise. In other words, the phrase “based on” describes both “based only on” and “based at least on.”

The methods disclosed herein comprise one or more steps or actions for achieving the described method. The method steps and/or actions may be interchanged with one another without departing from the scope of the present invention. In other words, unless a specific order of steps or actions is required for proper operation of the example, the order and/or use of specific steps and/or actions may be modified without departing from the scope of the present system and methods described herein.

While specific examples and applications of the present system and methods described herein have been illustrated and described, it is to be understood that the invention is not limited to the precise configuration and components disclosed herein. Various modifications, changes and variations, which will be apparent to those, skilled in the art may be made in the arrangement, operation, and details of the methods and systems of the present invention disclosed herein without departing from the spirit and scope of the invention.

What is claimed is:

1. A method for measuring and quantifying biological compounds, comprising illuminating a first side of a sample with a light source;
detecting light transmitted from a second side of the sample, wherein the second side of the sample is opposite the first side of the sample; and
obtaining a result based on the detected light.

2. The method of claim 1, wherein detecting the transmitted light comprises using an optical detector.

3. The method of claim 1, wherein the sample is one of:
skin;
fibrous tissue;
fat;
bone;
blood; and
cartilage.

4. The method of claim 1, wherein the sample is one of:
a finger;
a hand;
a tissue fold on an arm;
a tissue fold of a breast;
a tissue fold of a hand;
a thenar tissue fold; and
an earlobe.

5. The method of claim 1, wherein the light source has an intensity that does not substantially alter biological compound levels in the sample.

6. The method of claim 1, wherein the light source is one of a light emitting diode, a light emitting diode array, a tungsten-halogen lamp, and another suitable broad band light source.

7. The method of claim 1, wherein the result is based on levels of carotenoids in the sample.

8. The method of claim 7, wherein the light source generates light at a wavelength that overlaps the absorption band of carotenoids.

9. The method of claim 7, wherein the result is based on transmitted light detected in a spectral region centered at approximately 480 nm.

10. The method of claim 7, wherein obtaining a result comprises analyzing the detected light to obtain a result, and wherein the method further comprises displaying the result.

11. The method of claim 7, further comprising using the result to obtain an antioxidant status of the sample.

12. The method of claim 7, further comprising comparing concentration levels of carotenoids in the result to concentration levels of carotenoids in normal biological tissue to assess the risk or presence of a malignancy or other disease.

13. The method of claim 1, wherein the result is based on time-resolved absorption of the sample.

14. The method of claim 13, wherein the result of the time-resolved absorption of the sample is based on blood vessels in the sample expanding and contracting in rhythm with a human heartbeat.

15. The method of claim 14, wherein obtaining the result comprises analyzing the sample to determine carotenoid levels circulating in blood and carotenoid levels in the sample.

16. The method of claim 14, wherein obtaining the result comprises analyzing the sample to determine the level of other chromophores circulating in blood relative to their levels in the sample.

17. The method of claim 1, wherein the sample is approximately a millimeter to three centimeters thick, measuring from the first side of the sample to the second side of the sample.

18. An apparatus for measuring and quantifying biological compounds, comprising:
a light source that illuminates a first side of a sample; and
an optical detector that detects light transmitted from a second side of the sample, wherein the second side of the sample is opposite the first side of the sample.

19. The apparatus of claim 18, further comprising an enclosure, wherein the enclosure prevents the optical detector from detecting any light not transmitted from the second side of the sample.

20. The apparatus of claim 18, wherein the sample is one of:
skin;
fibrous tissue;
fat;
bone;
blood; and
cartilage.

21. The apparatus of claim 18, wherein the sample is one of:
a finger;
a hand;
a tissue fold of an arm;
a tissue fold of a breast;
a tissue fold of a hand;
a thenar tissue fold; and
an earlobe.

22. The apparatus of claim 18, wherein the light source has an intensity that does not substantially alter biological compound levels in the sample.

23. The apparatus of claim 18, wherein the light source is one of a light emitting diode, a light emitting diode array, a tungsten-halogen lamp, and another suitable broad band light source.

24. The apparatus of claim 18, further comprising a spectrograph/detector combination that analyses and quantifies the transmitted light detected at the optical detector to obtain a result.

25. The apparatus of claim 24, further comprising a display to display the result.

26. The apparatus of claim 24, wherein the result is based on levels of carotenoids in the sample.

27. The apparatus of claim 26, wherein the light source generates light at a wavelength that overlaps the absorption band of carotenoids.

28. The apparatus of claim 26, wherein the result is based on transmitted light detected at approximately 480 nm.

29. The apparatus of claim 26, further comprising using the result to obtain an antioxidant status of the sample.

30. The apparatus of claim 26, further comprising comparing concentration levels of carotenoids in the result to concentration levels of carotenoids in normal biological tissue to assess the risk or presence of a malignancy or other disease.

31. The apparatus of claim 18, wherein the result is based on time-resolved absorption in the sample.

32. The apparatus of claim 31, wherein the result of the time-resolved absorption of the sample is based on blood vessels in the sample expanding and contracting in rhythm with a human heartbeat.

33. The method of claim 32, wherein obtaining the result comprises analyzing the sample to determine carotenoid levels circulating in blood and carotenoid levels in the sample.

34. The method of claim 32, wherein obtaining the result comprises analyzing the sample to determine the level of other chromophores circulating in blood relative to their levels in the sample.

35. The apparatus of claim 18, wherein the sample is approximately a millimeter to three centimeters thick, measuring from the first side of the sample to the second side of the sample.
